(19) United States
(12) Patent Application Publication
(10) Pub. No.: US 2010/0198060 A1
(43) Pub. Date: Aug. 5, 2010

(54) Coupling of Powerhead Ram and Power Injector Syringe

(76) Inventors: Frank M. Fago, Mason, OH (US); Jonathan D. Gibbs, Mason, OH (US)

Correspondence Address: Mallinckrodt Inc., 675 McDonnell Boulevard, HAZELWOOD, MO, 63042, US

(21) Appl. No.: 12/669,496

(22) PCT Filed: Sep. 17, 2008
(86) PCT No.: PCT/US08/76593

§ 371 Date: Jan. 18, 2010

Related U.S. Application Data
(60) Provisional application No. 60/975,838, filed on Sep. 28, 2007.

(51) Int. Cl. A61B 6/00; A61M 5/32

(52) U.S. Cl. 600/432

(57) ABSTRACT

A ram assembly (110) for a power injector (10) is disclosed having an inner ram (120) that is movable relative to an outer ram (140). One or more ram couplers (158) each include a cam slot (164) and are slidably interconnected with an end (150) of the outer ram (140). The inner ram (120) includes a cam (128) that is disposed within a cam slot (164) of its corresponding ram coupler (158). Relative movement between the inner ram (120) and the outer ram (140) moves the various ram couplers (158) relative to the outer ram end (150) by the camming effect between the cams (128) and the cam slots (164). The ram couplers (158) may be used to establish both a coupled state and an uncoupled state or condition with a syringe plunger coupler (34) of a syringe plunger (32) for a power injector syringe (28).

### RELATED APPLICATIONS

This application claims priority to US provisional application Ser. No. 60/975,838 filed on 28 Sep. 2007 and entitled COUPLING OF POWERHEAD RAM AND POWER INJECTOR SYRINGE.

### FIELD OF THE INVENTION

The present invention generally relates to power injectors and, more particularly, to the manner of coupling/decoupling the power injectors syringe plunger drive system and syringe plunger.

### BACKGROUND

Various medical procedures require that one or more fluids be injected into the patient. Medical imaging procedures oftentimes involve the injection of a contrast media into the patient, possibly along with saline or other fluids. Other medical procedures involve injecting one or more fluids into a patient for therapeutic purposes. Power injectors may be used for these types of applications.

A power injector generally includes what is commonly referred to as a powerhead. One or more syringes may be mounted to the powerhead in various manners (e.g., detachably; rear-loading; front-loading). Each syringe typically includes what may be characterized as a syringe plunger, piston, or the like. Each such syringe plunger is appropriately interconnected with an appropriate syringe driver that is incorporated into the powerhead, such that operation of the syringe driver axially advances the associated syringe plunger. One typical syringe driver is in the form of a ram that mounted on a threaded lead or drive screw. Rotation of the drive screw in one rotational direction advances the associated ram in one axial direction, while rotation of the drive screw in the opposite rotational direction advances the associated ram in the opposite axial direction.

It is typical for one or more syringes to be mounted to a powerhead for an injection procedure, and to then be removed after completion of the injection procedure. There is therefore a need to provide a way to couple the syringe driver and the syringe plunger, and to thereafter decouple the syringe driver and syringe plunger. Various designs have been at least proposed to provide a detachable coupling between the syringe driver and syringe plunger, including: 1) moving the syringe away from the axis along with the syringe driver and syringe plunger move during an injection procedure in order to decouple the syringe driver and syringe plunger; 2) rotating the syringe relative to the syringe driver to dispose the components in position for coupling or decoupling of the syringe driver and syringe plunger, typically in combination with at least some type of relative axial between the syringe drive and syringe plunger; and 3) using pivotable jaws or the like on the syringe driver to couple with the syringe plunger, typically in combination with at least some type of relative axial between the syringe drive and syringe plunger, with a relative movement between the syringe drive and syringe plunger within a plane that is orthogonal to the axis along which the syringe driver and syringe plunger moving during an injection procedure, or both.

### SUMMARY

A first aspect of the present invention is embodied by a ram assembly for a power injector that is movable along a path to discharge a fluid from a syringe when interconnected with the ram assembly. Components of this ram assembly include a first ram section in the form of an inner ram, a second ram section in the form of an outer ram, and a ram coupler. The outer ram is movable relative to the inner ram. Moreover, the ram coupler is movable relative to at least one of the inner ram and outer ram in response to a movement of the outer ram relative to the inner ram. This movement of the ram coupler either couples or decouples the ram coupler and a syringe plunger coupler of the above-noted syringe, or stated another way changes the coupled state between the ram coupler and the associated syringe plunger coupler.

A second aspect of the present invention is embodied by a ram assembly for a power injector that is movable along a path to discharge a fluid from a syringe when interconnected with the ram assembly. Components of this ram assembly include a first ram section in the form of an inner ram, a second ram section in the form of an outer ram that includes an outer ram end that defines an end of the ram assembly, and a ram coupler. The outer ram is rotatable relative to the inner ram. Moreover, the ram coupler is slidably interconnected with the outer ram such that a movement of the ram coupler relative to the outer ram either couples or decouples the ram coupler and a syringe plunger coupler of the above-noted syringe, or stated another way changes the coupled state between the ram coupler and the associated syringe plunger coupler. This type of movement of the ram coupler is in response to a relative rotational movement between the inner and outer rams.

A third aspect of the present invention is embodied by a method for changing a coupled state between a syringe and a ram assembly of a power injector. The syringe includes a plunger, that in turn includes a syringe plunger coupler that is interconnected and movable along with the syringe plunger. The ram assembly includes a first ram section, a second ram section, and a ram coupler. The first ram section is rotated relative to the second ram section. The ram coupler is moved relative to at least one of the first and second ram sections in response to the relative rotational movement between the first and second ram sections so as to change a coupled state between the syringe plunger coupler and the ram coupler.

Various refinements exist of the features noted in relation to each of the above-noted first, second, and third aspects of the present invention. Further features may also be incorporated in each of the above-noted first, second, and third aspects of the present invention as well. These refinements and additional features may exist individually or in any combination in relation to each of the first, second, and third aspects. That is, each of the following features that will be discussed in relation to the first, second, and third aspects are not required to be used with any other feature or combination of features unless clearly noted to the contrary.

The ram assembly may be incorporated in any appropriate manner by any appropriate power injector. Such a power injector may be used for any appropriate application where the delivery of one or more fluids is desired, including without limitation any appropriate medical application (e.g., computed tomography or CT imaging; magnetic resonance imaging or MRI; SPECT imaging; PET imaging; X-ray imaging; angiographic imaging; optical imaging; ultrasound imaging). The power injector may be used in conjunction with any component or combination of components, such as an appropriate imaging system (e.g., a CT scanner). For instance, information could be conveyed between the power injector and one or more other components (e.g., scan delay information, injection start signal, injection rate). Any appropriate number of syringes may be integrated with the power injector in any appropriate manner (e.g., detachably; front-loaded; rear-loaded), any appropriate fluid may be discharged from a given syringe of the power injector, and any appropriate fluid may be discharged from a multiple syringe power injector configuration in any appropriate manner (e.g., sequentially, simultaneously), or any combination thereof. In one embodiment, fluid discharged from a syringe by operation of the power injector is directed into a conduit, where this conduit is fluidly interconnected with the syringe in any appropriate manner and directs fluid to a desired location (e.g., to a patient).

The first ram section (e.g., inner ram), second ram section (e.g., outer ram), and ram coupler may collectively move along an axial path. Such a movement of the ram assembly may provide and/or accommodate various functions. In one embodiment, the ram assembly is moved along an axial path to discharge a fluid from an interconnected syringe (e.g., a discharge stroke). In one embodiment, the ram assembly may be moved along an axial path into a position for subsequent execution of a discharge stroke. In one embodiment, the ram assembly may be moved along an axial path for purposes of loading or at least accommodating a loading of a fluid into an interconnected syringe (e.g., to dispose a syringe plunger in a retracted position). Generally, an axial movement of the ram assembly in any given direction and/or manner may provide any appropriate function or combination of functions.

Each of the first ram section (e.g., inner ram) and second ram section (e.g., outer ram) may be of any appropriate size, shape, configuration, and/or type. In one embodiment, the second ram section is disposed about at least part of the first ram section. In one embodiment, a first ram section in the form of an inner ram may be at least partially disposed within a second ram section in the form of an outer ram. In one embodiment, the first and second ram sections are concentrically disposed.

Relative movement between the first ram section (e.g., inner ram) and second ram section (e.g., outer ram) may be allowed in one dimension (e.g., relative rotational movement may be allowed between the inner and outer ram, and whether the inner ram is rotated, the outer ram is rotated, or both), while relative movement between the first and second ram sections in at least one other dimension may be restrained (e.g., the first and second ram sections may be maintained in an at least substantially fixed positional relationship during an axial movement of the ram assembly). A relative movement between the first and second ram sections that actuates one or more ram couplers may be of any appropriate type or in any appropriate dimension (e.g., a rotational motion), and this relative movement may be realized in any appropriate manner. Only a certain relative movement is required between the first and second ram sections. Therefore the second ram section may be moved in a certain dimension to actuate one or more ram couplers, while the first ram section is maintained in a fixed position in this same dimension during actuation of the ram coupler(s), or vice versa.

At least a certain relative movement between the first ram section (e.g., inner ram) and the second ram section (e.g., outer ram) may be used to actuate one or more ram couplers to change a coupled state between the ram coupler(s) and an associated syringe coupler. Changing a “coupled state” again encompasses both establishing a coupling between the ram coupler with a syringe plunger coupler, and decoupling the ram coupler from its associated syringe plunger coupler. For instance, the ram coupler may move relative to the first ram section, the second ram section, or both to change its coupled state with an associated syringe plunger coupler. The relative movement between the first and second ram sections and a relative movement between the ram coupler and at least one of the first and second ram sections to change its coupled state with a syringe plunger coupler may be of different types or in different dimensions. In one embodiment, the first and/or second ram section rotate to actuate an axial movement of the ram coupler relative to at least one of the first and second ram sections.

The ram coupler may be of any appropriate size, shape, configuration, and/or type. Any appropriate motion in one or more dimensions may be utilized by the ram coupler to change its coupled state with an associated syringe coupler. In one embodiment, the ram coupler moves along an axial path relative to at least one of the first ram section (e.g., inner ram) and the second ram section (e.g., outer ram) to change a coupled state with an associated syringe plunger coupler. In one embodiment, the ram coupler may move at least generally away from an axis (e.g., along with the entire ram assembly may move) to achieve one of a coupled or decoupled state with an associated syringe plunger coupler, and may move at least generally toward such this same axis to achieve the other of the coupled or decoupled state. In each of these instances, the ram coupler may be slidably interconnected with either the first ram section or the second ram section.

Any appropriate way of integrating the ram coupler with the ram assembly may be utilized. In one embodiment, the ram coupler is slidably interconnected with a second ram section in the form of an outer ram. For instance, the outer ram may include a slot, and the ram coupler may be disposed within this slot. In one embodiment, an end of a second ram section in the form of an outer ram includes a slot for the ram coupler, where this outer ram end projects toward a corresponding syringe when interconnected therewith.

A camming action may be utilized to produce a relative movement of the ram coupler to change its coupled state with an associated syringe plunger coupler. In one embodiment, one of the first ram section (e.g., inner ram) and the second ram section (e.g., outer ram) includes what may be characterized as a first camming member or element (e.g., a cam), while the ram coupler may included a second camming member or element (e.g., a cam follower). Each of these camming members may be of any appropriate size, shape, configuration, and/or type.

Multiple ram couplers may be used by the ram assembly, and multiple ram couplers may be disposed in any appropriate arrangement. The entirety of the discussion presented herein with regard to a ram coupler may apply to each ram coupler in a multiple ram coupler configuration as desired/required. Multiple ram couplers may collectively move to change a coupled state with an associated syringe plunger coupler. Each of a plurality of ram couplers may be movable at least generally away from a common locale, may be movable at least generally toward a common locale, or both to affect a change in a coupled state with an associate syringe plunger coupler. In one embodiment, this common locale is a central, longitudinal reference axis that coincides with a length dimension of the ram assembly, or stated another way along which the ram assembly may move. In one embodiment, each of a plurality of ram couplers are movable along a different axial path relative to at least one of the first and second ram sections (e.g., inner and outer rams) to change a coupled state with an associated syringe plunger coupler.

In a first embodiment, the first ram section may be in the form of an inner ram, the second ram section may be in the form of an outer ram, the inner ram may be a least partially disposed within the outer ram, the outer ram may include an end that defines an end of the ram assembly, and the ram coupler may be slidably interconnected with this outer ram end. Various characterizations may be made in relation to this first embodiment, will now be addressed, and apply individually and in any desired combination.

The above-noted first embodiment may include a rotational lock that is engaged with the outer ram such that the inner ram may be rotated to move the ram coupler relative to the outer ram end. In this case, the outer ram may be maintained at least generally in a rotationally stationary position by the rotational lock. The range through which the inner ram may be rotated in this instance may be limited in any appropriate manner. For instance, the outer ram may include a slot. A stop may extend through this slot to the inner ram and this stop may be maintained in a fixed position relative to the inner ram in any appropriate manner (e.g., the stop may be mounted to the inner ram in any appropriate manner). During rotation of the inner ram, the stop may advance along the outer ram slot. The length of the outer ram slot may thereby establish the range through which the inner ram may be rotated.

The above-noted first embodiment may include a rotational lock that is interconnected with the inner ram (versus the outer ram as noted above) such that the outer ram may be rotated to move the ram coupler relative to the outer ram end. In this case, the inner ram may be maintained at least generally in a rotationally stationary position by the rotational lock.

The above-noted first embodiment may have the ram coupler be slidably interconnected with the outer ram end. For instance, the outer ram end may include a slot, and the ram coupler may be slidably disposed within this slot. In any case, the inner ram may include a first camming member or element, and the ram coupler may include a second camming member or element that is at least engageable with the first camming member. Relative movement between the first and second camming members (e.g., by moving the inner ram relative to the outer ram) may actuate the ram coupler (e.g., move the ram coupler relative to the outer ram). In one embodiment, the first camming member is disposed on an end of the inner ram, while the second camming member is disposed on a surface of the ram coupler that projects toward this inner ram end. Each of the first and second camming members may be of any appropriate size, shape, configuration, and/or type in accordance with the foregoing.

The ram coupler may be disposed in a position so that a head of a syringe plunger coupler is in a captured state during a retraction of the ram assembly, during an extension of the ram assembly (e.g., for advancing a syringe plunger of a syringe on a discharge stroke), or both. This syringe plunger coupler may extend from a syringe plunger, and the ram coupler may be disposed between this head and the syringe plunger and in alignment with the head in a dimension that the ram assembly moves, to in turn move the syringe plunger. The ram coupler may also be disposed in a position so that an end of the inner ram engages the head of the syringe plunger coupler during an extension of the ram assembly (e.g., for advancing a syringe plunger of a syringe on a discharge stroke). For instance, the ram coupler may be moved so as to be out of alignment with the head of the syringe plunger coupler in a dimension that the ram assembly moves, to in turn move the syringe plunger.

The ram assembly may be incorporated by a power injector of any appropriate size, shape, configuration, and/or type. This power injector may include a rotatable drive screw, and the ram assembly may be interconnected with this drive screw to move along the drive screw by rotating the drive screw. The direction that the ram assembly moves along the drive screw may depend upon the rotational direction of the drive screw. The inner ram, the outer ram, or both may be rotated to actuate the ram coupler. In one configuration, a corresponding syringe and the outer ram may be coupled such that rotation of the syringe (e.g., where this rotational motion may mount or disconnect the syringe from a powerhead of the power injector) in turn rotates the outer ram to actuate the ram coupler. In another configuration, rotation of the drive screw may rotate the inner ram to actuate the ram coupler, and the amount that the inner ram is allowed to rotate may be limited in accordance with the foregoing. The inner ram may rotate to actuate the ram coupler (e.g., the amount that the inner ram is allowed to rotate may be limited in accordance with the foregoing), and this rotation of the inner ram may be in response to a rotation of the drive screw. A threaded interconnection between the drive screw and the ram assembly, along with rotation of the drive screw, may provide the force that is used to rotate the inner ram along with the drive screw. In each of these embodiments, each of the inner ram and ram coupler may include interacting camming members or elements in accordance with the foregoing.

### BRIEF DESCRIPTION OF THE FIGURES

FIG. 1 is a schematic of one embodiment of a power injector.

FIG. 2A is a perspective view of one embodiment of a portable stand-mounted, dual-head power injector.

FIG. 2B is an enlarged, partially exploded, perspective view of a powerhead used by the power injector of FIG. 2A.

FIG. 2C is a schematic of one embodiment of a syringe plunger drive assembly used by the power injector of FIG. 2A.

FIG. 3A is an exploded, perspective view of one embodiment of a ram assembly that utilizes a rotatable section for actuating a coupler to engage a syringe plunger coupler, and that may be utilized by a power injector.

FIG. 3B is a plan view of one of the ram couplers from the ram assembly of FIG. 3A, illustrating its cam slot.

FIG. 4A is an end view of the ram assembly of FIG. 3A, where the ram couplers are in position for retracting a syringe plunger.

FIG. 4B is an end view of the ram assembly of FIG. 3A, where the ram couplers are in position for advancing a syringe plunger for a fluid discharge or a discharge stroke

FIG. 5 is an exploded, perspective view of a variation of the ram assembly of FIG. 3A.

FIG. 6A is an end view of the ram assembly of FIG. 5, where the ram couplers are in position for being coupled with and advancing a syringe plunger.

FIG. 6B is an end view of the ram assembly of FIG. 3A, where the ram couplers are in position for being uncoupled from the syringe plunger.

FIG. 7 is an exploded, perspective view of one embodiment for providing an actuating force to move the ram couplers from the FIG. 6A position (coupled/coupling) to the FIG. 6B position (uncoupled/uncoupling).

FIG. 8 is another perspective view of the faceplate illustrated in FIG. 7.

### DETAILED DESCRIPTION

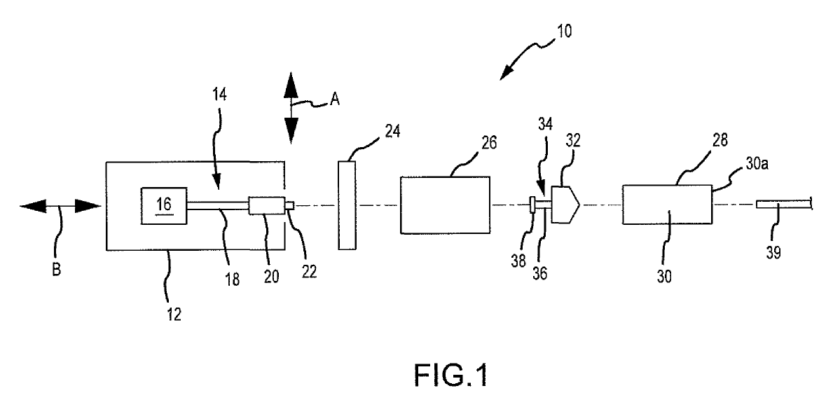

FIG. 1 presents a schematic of one embodiment of a power injector 10 having a powerhead 12. A syringe 28 may be installed on this powerhead 12 and may be considered to be part of the power injector 10. Some injection procedures may result in a relatively high pressure being generated within the syringe 28. In this regard, it may be desirable to dispose the syringe 28 within a pressure jacket 26. The pressure jacket 26 is typically installed on the powerhead 12, followed by disposing the syringe 28 within the pressure jacket 26. The same pressure jacket 26 will typically remain installed on the powerhead 12, as various syringes 28 are positioned within and removed from the pressure jacket 26 for multiple injection procedures. The power injector 10 may eliminate the pressure jacket 26 if the power injector 10 is configured/utilized for low-pressure injections. In any case, fluid discharged from the syringe 28 may be directed into a conduit 39 of any appropriate size, shape, configuration, and/or type, which may be fluidly interconnected with the syringe 28 in any appropriate manner, and which may direct fluid to any appropriate location (e.g., to a patient).

The powerhead 12 includes a syringe plunger drive assembly 14 that interfaces with the syringe 28 to discharge fluid from the syringe 28. This syringe plunger drive assembly 14 includes a drive source 16 (e.g., a motor of any appropriate size, shape, configuration, and/or type, optional gearing, and the like) that powers a drive output 18 (e.g., a rotatable drive screw). A ram 20 may be advanced along an appropriate path (e.g., axial) by the drive output 18. The ram 20 may include a coupler 22 for interfacing with a corresponding portion of the syringe 28 in a manner that will be discussed below.

The syringe 28 includes a plunger or piston 32 that is movably disposed within a syringe barrel 30 (e.g., for axial reciprocation along an axis coinciding with the double-headed arrow B). The plunger 32 may include a coupler 34. This syringe plunger coupler 34 may interconnect with the ram coupler 22 to allow the syringe plunger drive assembly 14 to retract the syringe plunger 32 within the syringe barrel 30. Retraction of the syringe plunger 32 may be utilized to accommodate a loading of fluid into the syringe barrel 30 for a subsequent injection or discharge, may be utilized to actually draw fluid into the syringe barrel 30 for a subsequent injection or discharge, or for any other appropriate purpose. Certain configurations may not require that the syringe plunger drive assembly 14 be able to retract the syringe plunger 32, in which case the ram coupler 20 and syringe plunger coupler 34 may not be required. Even when a ram coupler 22 and syringe plunger coupler 32 are utilized, it may such that these components may or may not be coupled when the ram 20 advances the syringe plunger 32 to discharge fluid from the syringe 28 (e.g., the ram 20 may simply “push on” the syringe plunger 34). Any single motion or combination of motions in any appropriate dimension or combination of dimensions may be utilized to dispose the ram coupler 22 and syringe plunger coupler 34 in a coupled state or condition, to dispose the ram coupler 22 and syringe plunger coupler 34 in an un-coupled state or condition, or both.

The syringe 28 may be installed on the powerhead 12 in any appropriate manner. For instance, the syringe 28 could be configured to be installed directly on the powerhead 12. In the illustrated embodiment, a housing 24 is appropriately mounted on the powerhead 12 to provide an interface between the syringe 28 and the powerhead 12. This housing 24 may be in the form of an adapter to which one or more configurations of syringes 28 may be installed, and where at least one configuration for a syringe 28 could be installed directly on the powerhead 12 without using any such adapter. The housing 24 may also be in the form of a faceplate to which one or more configurations of syringes 28 may be installed. In this case, it may be such that a faceplate is required to install a syringe 28 on the powerhead 12—the syringe 28 could not be installed on the powerhead 12 without the faceplate. When a pressure jacket 26 is being used, it may be installed on the powerhead 12 in the various manners discussed herein in relation to the syringe 28, and the syringe 28 will then thereafter be installed in the pressure jacket 26.

The housing 24 may be mounted on and remain in a fixed position relative to the powerhead 12 when installing a syringe 28. Another option is to movably interconnect the housing 24 and the powerhead 12 to accommodate installing a syringe 28. For instance, the housing 24 may move within a plane that contains the double-headed arrow A to provide one or more of coupled state or condition and an un-coupled state or condition between the ram coupler 22 and the syringe plunger coupler 34.

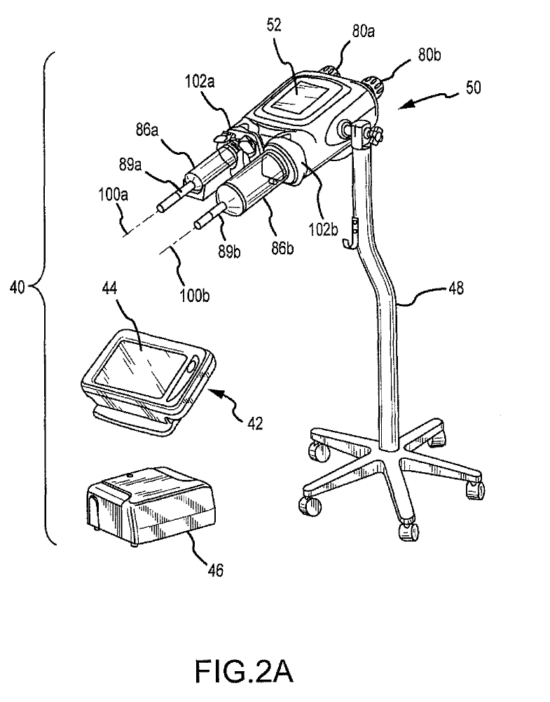

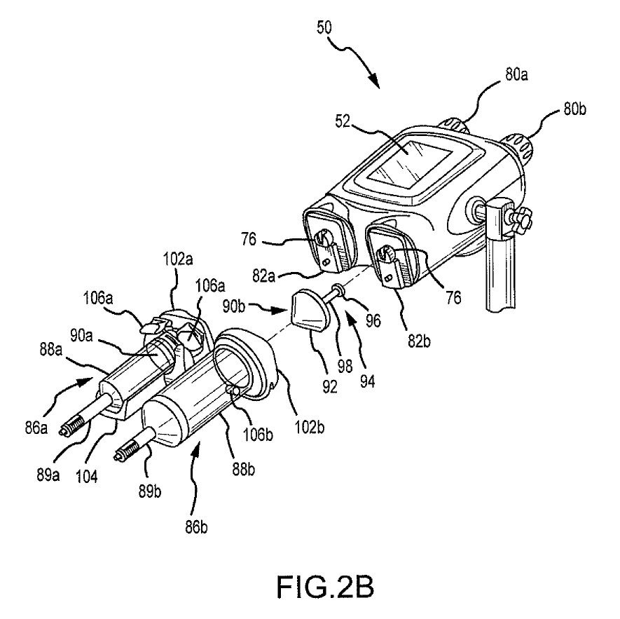

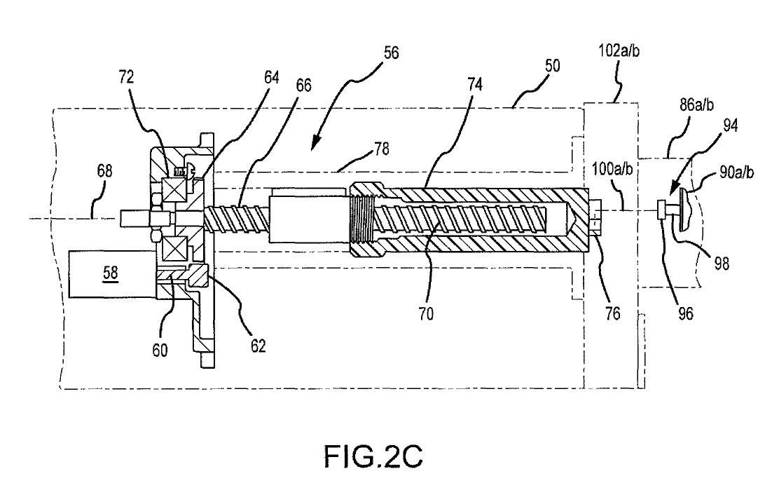

One particular power injector configuration is illustrated in FIG. 2A, is identified by a reference numeral 40, and is at least generally in accordance with the power injector 10 of FIG. 1. The power injector 40 includes a powerhead 50 that is mounted on a portable stand 48. A pair of syringes 86a, 86b for the power injector 40 is mounted on the powerhead 50. Fluid may be discharged from the syringes 86a, 86b during operation of the power injector 40.

The portable stand 48 may be of any appropriate size, shape, configuration, and/or type. Wheels, rollers, casters, or the like may be utilized to make the stand 48 portable. The powerhead 50 could be maintained in a fixed position relative to the portable stand 48. However, it may be desirable to allow the position of the powerhead 50 to be adjustable relative to the portable stand 48 in at least some manner. For instance, it may be desirable to have the powerhead 50 in one position relative to the portable stand 48 when loading fluid into one or more of the syringes 86a, 86b, and to have the powerhead 50 in a different position relative to the portable stand 48 for performance of an injection procedure. In this regard, the powerhead 50 may be movably interconnected with the portable stand 48 in any appropriate manner (e.g., such that the powerhead 50 may be pivoted through at least a certain range of motion, and thereafter maintained in the desired position).

It should be appreciated that the powerhead 50 could be supported in any appropriate manner for providing fluid. For instance, instead of being mounted on a portable structure, the powerhead 50 could be interconnected with a support assembly, that in turn is mounted to an appropriate structure (e.g., ceiling, wall, floor). Any support assembly for the powerhead 50 may be positionally adjustable in at least some respect (e.g., by having one or more support sections that may be repositioned relative to one more other support sections), or may be maintained in a fixed position. Moreover, the powerhead 50 may be integrated with any such support assembly so as to either be maintained in a fixed position or so as to be adjustable relative the support assembly.

The powerhead 50 includes a graphical user interface or GUI 52. This GUI 52 may be configured to provide one or any combination of the following functions: controlling one or more aspects of the operation of the power injector 40; inputting/editing one or more parameters associated with the operation of the power injector 40; and displaying appropriate information (e.g., associated with the operation of the power injector 40). The power injector 40 may also include a console 42 and powerpack 46 that each may be in communication with the powerhead 50 in any appropriate manner (e.g., via one or more cables), that may be placed on a table or mounted on an electronics rack in an examination room or at any other appropriate location, or both. The powerpack 46 may include one or more of the following and in any appropriate combination: a power supply for the injector 40; interface circuitry for providing communication between the console 42 and powerhead 50; circuitry for permitting to connection of the power injector 40 to remote units such as remote consoles, remote hand or foot control switches, or other original equipment manufacturer (OEM) remote control connections (e.g., to allow for the operation of injector 20 to be synchronized with the x-ray exposure of an imaging system); and any other appropriate componentry. The console 42 may include a touch screen display 44, which in turn may provide one or more of the following functions and in any appropriate combination: allowing an operator to remotely control one or more aspects of the operation of the power injector 40; allowing an operator to enter/edit one or more parameters associated with the operation of the power injector 40; allowing an operator to specify and store programs for automated operation of the power injector 40 (which can later be automatically executed by the power injector 40 upon initiation by the operator); and displaying any appropriate information relation to the power injector 40 and including any aspect of its operation.

Various details regarding the integration of the syringes 86a, 86b with the powerhead 50 are presented in FIG. 2B. Each of the syringes 86a, 86b includes the same general components. The syringe 86a includes plunger or piston 90a that is movably disposed within a syringe barrel 88a. Movement of the plunger 90a along an axis 100a (FIG. 2A) via operation of the powerhead 50 will discharge fluid from within the syringe barrel 88a through a nozzle 89a of the syringe 86a. An appropriate conduit (not shown) will typically be fluidly interconnected with the nozzle 89a in any appropriate manner to direct fluid to a desired location (e.g., a patient). Similarly, the syringe 86b includes plunger or piston 90b that is movably disposed within a syringe barrel 88b. Movement of the plunger 90b along an axis 100b (FIG. 2A) via operation of the powerhead 50 will discharge fluid from within the syringe barrel 88b through a nozzle 89b of the syringe 86b. An appropriate conduit (not shown) will typically be fluidly interconnected with the nozzle 89b in any appropriate manner to direct fluid to a desired location (e.g., a patient).

The syringe 86a is interconnected with the powerhead 50 via an intermediate faceplate 102a. This faceplate 102a includes a cradle 104 that supports at least part of the syringe barrel 88a, and which may provide/accommodate any additional functionality or combination of functionalities. A mounting 82a is disposed on and is fixed relative to the powerhead 50 for interfacing with the faceplate 102a. A ram coupler 76 of a ram 74, which are each part of a syringe plunger drive assembly 56 for the syringe 86a, is positioned in proximity to the faceplate 102a when mounted on the powerhead 50. Details regarding the syringe plunger drive assembly 56 will be discussed in more detail below in relation to FIG. 2C. Generally, the ram coupler 76 may be coupled with the syringe plunger 90a of the syringe 86a, and the ram coupler 76 and ram 74 may then be moved relative to the powerhead 50 to move the syringe plunger 90a along the axis 100a (FIG. 2A). It may be such that the ram coupler 76 is engaged with, but not actually coupled to, the syringe plunger 90a when moving the syringe plunger 90a to discharge fluid through the nozzle 89a of the syringe 86a.

The faceplate 104a may be moved at least generally within a plane that is orthogonal to the axes 100a, 100b (associated with movement of the syringe plungers 90a, 90b, respectively, and illustrated in FIG. 2A), both to mount the faceplate 104a on and remove the faceplate 104a from its mounting 82a on the powerhead 50. The faceplate 104a may be used to couple the syringe plunger 90a with its corresponding ram coupler 76 on the powerhead 50. In this regard, the faceplate 104a includes a pair of handles 106a. Generally and with the syringe 86a being initially positioned within the faceplate 102a, the handles 106a may be moved to in turn move/translate the syringe 86a at least generally within a plane that is orthogonal to the axes 100a, 100b (associated with movement of the syringe plungers 90a, 90b, respectively, and illustrated in FIG. 2A). Moving the handles 106a to one position moves/translates the syringe 86a (relative to the faceplate 102a) in an at least generally downward direction to couple its syringe plunger 90a with its corresponding ram coupler 76. Moving the handles 106a to another position moves/translates the syringe 86a (relative to the faceplate 102a) in an at least generally upward direction to uncouple its syringe plunger 90a from its corresponding ram coupler 76.

The syringe 86b is interconnected with the powerhead 50 via an intermediate faceplate 102b. A mounting 82b is disposed on and is fixed relative to the powerhead 50 for interfacing with the faceplate 102b. A ram coupler 76 of a ram 74, which are each part of a syringe plunger drive assembly 56 for the syringe 86b, is positioned in proximity to the faceplate 102b when mounted to the powerhead 50. Details regarding the syringe plunger drive assembly 56 again will be discussed in more detail below in relation to FIG. 2C. Generally, the ram coupler 76 may be coupled with the syringe plunger 90b of the syringe 86b, and the ram coupler 76 and ram 74 may be moved relative to the powerhead 50 to move the syringe plunger 90b along the axis 100b (FIG. 2A). It may be such that the ram coupler 76 is engaged with, but not actually coupled to, the syringe plunger 90b when moving the syringe plunger 90a to discharge fluid through the nozzle 89b of the syringe 86b.

The faceplate 104b may be moved at least generally within a plane that is orthogonal to the axes 100a, 100b (associated with movement of the syringe plungers 90a, 90b, respectively, and illustrated in FIG. 2A), both to mount the faceplate 104b on and remove the faceplate 104b from its mounting 82b on the powerhead 50. The faceplate 104b also may be used to couple the syringe plunger 90b with its corresponding ram coupler 76 on the powerhead 50. In this regard, the faceplate 104b may include a handle 106b. Generally and with the syringe 86b being initially positioned within the faceplate 102b, the syringe 86b may be rotated along its long axis 100b (FIG. 2A) and relative to the faceplate 102b. This rotation may be realized by moving the handle 106a, by grasping and turning the syringe 86b, or both. In any case, this rotation moves/translates both the syringe 86b and the faceplate 102b at least generally within a plane that is orthogonal to the axes 100a, 100b (associated with movement of the syringe plungers 90a, 90b, respectively, and illustrated in FIG. 2A). Rotating the syringe 86b in one direction moves/translates the syringe 86b and faceplate 102b in an at least generally downward direction to couple the syringe plunger 90b with its corresponding ram coupler 76. Rotating the syringe 86b in the opposite direction moves/translates the syringe 86b and faceplate 102b in an at least generally upward direction to uncouple its syringe plunger 90b from its corresponding ram coupler 76.

As illustrated in FIG. 2B, the syringe plunger 90b includes a plunger body 92 and a syringe plunger coupler 94. This syringe plunger coupler 94 includes a shaft 98 that extends from the plunger body 92, along with a head 96 that is spaced from the plunger body 92. Each of the ram couplers 76 includes a larger slot that is positioned behind a smaller slot on the face of the ram coupler 76. The head 96 of the syringe plunger coupler 94 may be positioned within the larger slot of the ram coupler 76, and the shaft 98 of the syringe plunger coupler 94 may extend through the smaller slot on the face of the ram coupler 76 when the syringe plunger 90b and its corresponding ram coupler 76 are in a coupled state or condition. The syringe plunger 90a may include a similar syringe plunger coupler 94 for interfacing with its corresponding ram coupler 76.

The powerhead 50 is utilized to discharge fluid from the syringes 86a, 86b in the case of the power injector 40. That is, the powerhead 50 provides the motive force to discharge fluid from each of the syringes 86a, 86b. One embodiment of what may be characterized as a syringe plunger drive assembly is illustrated in FIG. 2C, is identified by reference numeral 56, and may be utilized by the powerhead 50 to discharge fluid from each of the syringes 86a, 86b. A separate syringe plunger drive assembly 56 may be incorporated into the powerhead 50 for each of the syringes 86a, 86b. In this regard and referring back to FIGS. 2A-B, the powerhead 50 may include hand-operated knobs 29a and 29b for use in separately controlling each of the syringe plunger drive assemblies 56.

Initially and in relation to the syringe plunger drive assembly 56 of FIG. 2C, each of its individual components may be of any appropriate size, shape, configuration and/or type. The syringe plunger drive assembly 56 includes a motor 58, which has an output shaft 60. A drive gear 62 is mounted on and rotates with the output shaft 60 of the motor 58. The drive gear 62 is engaged or is at least engageable with a driven gear 64. This driven gear 64 is mounted on and rotates with a drive screw or shaft 66. The axis about which the drive screw 66 rotates is identified by reference numeral 68. One or more bearings 72 appropriately support the drive screw 66.

A carriage or ram 74 is movably mounted on the drive screw 66. Generally, rotation of the drive screw 66 in one direction axially advances the ram 74 along the drive screw 66 (and thereby along axis 68) in the direction of the corresponding syringe 86a/b, while rotation of the drive screw 66 in the opposite direction axially advances the ram 74 along the drive screw 66 (and thereby along axis 68) away from the corresponding syringe 86a/b. In this regard, the perimeter of at least part of the drive screw 66 includes helical threads 70 that interface with at least part of the ram 74. The ram 74 is also movably mounted within an appropriate bushing 78 that does not allow the ram 74 to rotate during a rotation of the drive screw 66. Therefore, the rotation of the drive screw 66 provides for an axial movement of the ram 74 in a direction determined by the rotational direction of the drive screw 66.

The ram 74 includes a coupler 76 that that may be detachably coupled with a syringe plunger coupler 94 of the syringe plunger 90a/b of the corresponding syringe 86a/b. When the ram coupler 76 and syringe coupler plunger 94 are appropriately coupled, the syringe plunger 90a/b moves along with ram 74. FIG. 2C illustrates a configuration where the syringe 86a/b may be moved along its corresponding axis 100a/b without being coupled to the ram 74. When the syringe 86a/b is moved along its corresponding axis 100a/b such that the head 96 of its syringe plunger 90a/b is aligned with the ram coupler 76, but with the axes 68 still in the offset configuration of FIG. 2C, the syringe 86a/b may be translated within a plane that is orthogonal to the axis 68 along which the ram 74 moves. This establishes a coupled engagement between the ram coupler 76 and the syringe plunger coupler 96 in the above-noted manner.

The power injectors 10, 40 of FIGS. 1 and 2A-C each may be used for any appropriate application, including without limitation for medical imaging applications where fluid is injected into a subject (e.g., a patient). Representative medical imaging applications for the power injectors 10, 40 include without limitation computed tomography or CT imaging, magnetic resonance imaging or MRI, SPECT imaging, PET imaging, X-ray imaging, angiographic imaging, optical imaging, and ultrasound imaging. The power injectors 10, 40 each could be used alone or in combination with one or more other components. The power injectors 10, 40 each may be operatively interconnected with one or more components, for instance so that information may be conveyed between the power injector 10, 40 and one or more other components (e.g., scan delay information, injection start signal, injection rate).

Any number of syringes may be utilized by each of the power injectors 10, 40, including without limitation single-head configurations (for a single syringe) and dual-head configurations (for two syringes). In the case of a multiple syringe configuration, each power injector 10, 40 may discharge fluid from the various syringes in any appropriate manner and according to any timing sequence (e.g., sequential discharges from two or more syringes, simultaneous discharges from two or more syringes, or any combination thereof). Each such syringe utilized by each of the power injectors 10, 40 may include any appropriate fluid, for instance contrast media, a radiopharmaceutical, or saline. Each such syringe utilized by each of the power injectors 10, 40 may be installed in any appropriate manner (e.g., rear-loading configurations may be utilized; front-loading configurations may be utilized).

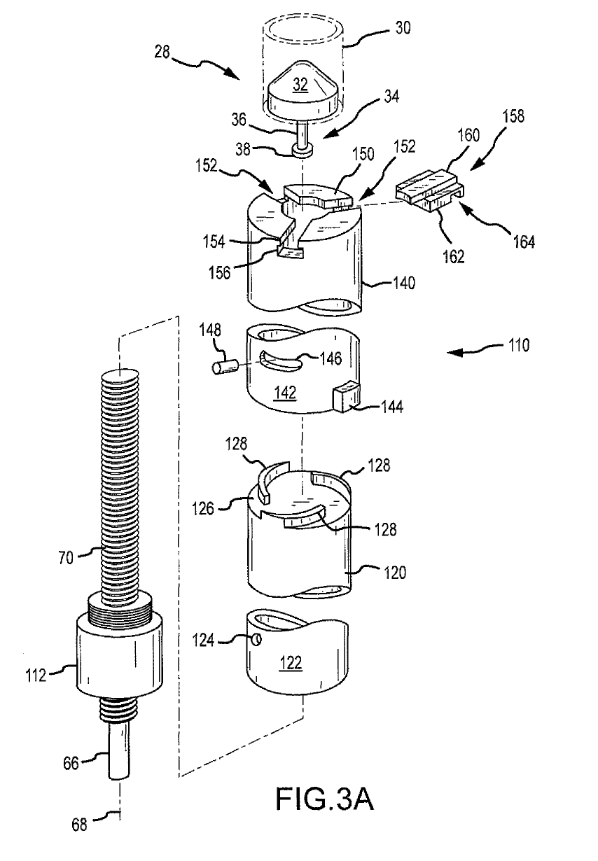

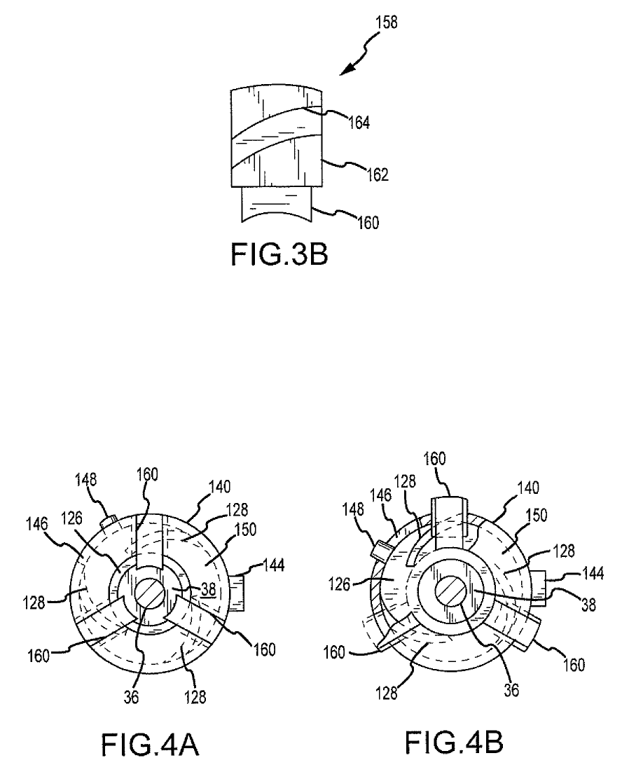

One embodiment of a ram assembly is illustrated in FIGS. 3A-B, is identified by reference 110, and may be used by the power injector 10 of FIG. 1 in place of the ram 20, as well as by the power injector 40 of FIG. 2A in place of the ram 74. However, it should be appreciated that the ram assembly 110 may be utilized by any appropriate power injector, and where this power injector may be utilized for any appropriate application. Hereafter, the ram assembly 110 will be described in relation to being incorporated by the power injector 10 of FIG. 1.

The ram assembly 110 includes a collar 112 that is threadably mounted on a drive screw 66 having helical threads 70. The drive output 18 for the power injector 10 of FIG. 1 would thereby be in the form of the drive screw 66 from the syringe plunger drive assembly 56 discussed above in relation to FIG. 2C. Rotation of the drive screw 66 in one rotational direction may advance the collar 112 along the drive screw 66 (and thereby along axis 68) in one axial direction. Rotation of the drive screw 66 in another rotational direction may advance the collar 112 along the drive screw 66 (and thereby along axis 68) in the opposite axial direction.

Another component of the ram assembly 110 is an inner ram 120, which may be mounted on the collar 112 in any appropriate manner (e.g., by a threaded engagement with at least a portion of the collar 112). The inner ram 120 could also be configured to include the collar 112 as an integral portion thereof, or the inner ram 120 and collar 112 could be separately fabricated, and fixed relative to each other in any appropriate manner. In any case, an aperture 124 extends at least partially through a sidewall 122 of the inner ram 120. In the illustrated embodiment, the sidewall 122 is cylindrical, although other shapes may be appropriate. A plurality of camming elements in the form of cams 128 is disposed on an end 126 of the inner ram 120. Each cam 128 may be of any appropriate size, shape, configuration, and/or type. One cam 128 is provided for each ram coupler 158 utilized by the ram assembly 110 and that will be addressed below. Since any appropriate number of ram couplers 158 may be utilized, correspondingly any appropriate number of cams 128 may be utilized as well. Multiple cams 128 may be disposed in any appropriate arrangement on the end 126 of the inner ram 120 that will move the ram couplers 158 between a coupling/coupled position and an uncoupling/uncoupled position.

The inner ram 120 may extend at least partially within an outer ram 140 of the ram assembly 110. Generally, the outer ram 140 and inner ram 120 may collectively move along the drive screw 66, and the inner ram 120 may rotate relative to the outer ram 140. In the illustrated embodiment, an anti-rotation key 144 is mounted to a sidewall 142 of the outer ram 140 in any appropriate manner. This anti-rotation key 144 may be of any appropriate size, shape, configuration and/or type that will allow the outer ram 140 to move axially along the drive screw 66 (together with the inner ram 120), and that will allow the inner ram 120 to rotate relative to the outer ram 140 at least generally about the drive screw 66. For instance, the anti-rotation key 144 could interface with an axially extending slot within the powerhead 12 (FIG. 1), such that the outer ram 140 does not rotate about or relative to the drive screw 66.

The inner ram 120 may rotate about the drive screw 66 to at least a certain degree to move the various ram couplers 158 relative to the outer ram 140. A rotation limit slot 146 extends through the sidewall 142 of the outer ram 140. A stop or pin 148 extends through this rotation limit slot 146 and into the aperture 124 on the sidewall 122 of the inner ram 120. The pin 148 could be mounted to or otherwise maintained in a fixed position relative to the sidewall 122 of the inner ram 120 in any appropriate manner. Based upon the foregoing, the outer ram 140 may be constrained so as to only be able to move axially along the drive screw 66 (together with the inner ram 120), while the inner ram 120 may be able to both move axially along the drive screw 66 and to rotate about the drive screw 66 through a certain rotational range established by the rotation limit slot 146 on the outer ram 140 and the pin 148 on the inner ram 120. Any appropriate way of providing a desired rotational range for the inner ram 120 may be utilized by the ram assembly 110.

The outer ram 140 includes an end 150. At least one ram coupler 158 is movably/slidably interconnected with the outer ram end 150. Any appropriate number of ram couplers 158 may be utilized by the ram assembly 110, including a single ram coupler 158 (not shown). Three ram couplers 158 are utilized in the illustrated embodiment. In any case, the outer ram end 150 includes a coupler slot 152 for each ram coupler 158 being utilized by the ram assembly 110. In the illustrated embodiment, each coupler slot 152 includes a first slot section 154 and a second slot section 156.

A ram coupler 158 may be movably/slidably disposed within a coupler slot 152 included on the outer ram end 150. Each ram coupler 158 may include: 1) a coupler section 160 that is at least partially disposed within the first slot section 154 of the corresponding coupler slot 152 on the outer ram end 150; and 2) a base 162 that is at least partially disposed within the second slot section 156 of the corresponding coupler slot 152 on the outer ram end 150. Generally, each ram coupler 158 may be moved within their corresponding coupler slot 152 in response to a relative movement between the inner ram 120 and the outer ram 140 (a relative rotational motion in the illustrated embodiment) between two general positions—a coupling or coupled position, along with an uncoupled or uncoupling position. In one embodiment, each ram coupler 158 moves along an axial path relative to the outer ram 140.

Referring now to FIG. 3B, the underside of the base 162 of each ram coupler 158 (that which is opposite of its corresponding coupler section 160) includes another camming element in the form of a cam slot 164. Each cam 128 on the inner ram end 126 is positioned within a cam slot 164 of a separate ram coupler 158. Movement of the ram couplers 158 within their respective coupler slots 152 and relative to the outer ram 140 is provided by or responsive to rotating the inner ram 120 relative to the outer ram 140. The interface between each cam 128 and its corresponding cam slot 164 moves the ram couplers 158 within their respective coupler slot 152 in a direction dictated by the rotational direction of the drive screw 66 in the illustrated embodiment. The camming elements included on the inner ram end 126 (the cams 128 in the illustrated embodiment), as well as the camming elements included on the base 162 of the ram couplers 158 (the cam slots 164 in the illustrated embodiment), each may be of any appropriate size, shape, configuration, and/or type. For instance, the cams 128 could be incorporated into the base 162 of the ram couplers 158 and the cam slots 164 could be incorporated into the inner ram end 126 (not shown). Moreover, the cams 128 each could be in the form of a simple pin versus the illustrated arcuate segments.

FIGS. 4A and 4B illustrate two general positions for the ram couplers 158. The ram couplers 158 are positioned in FIG. 4A to engage and pull on the head 38 of the corresponding syringe plunger coupler 34 during retraction of the ram assembly 110 along the drive screw 66. This may be referred to as a coupled or coupling position for the ram couplers 158. One characterization of the coupled or coupling position is that the ram couplers 158 are moved at least generally toward the axis 68 along which the ram assembly 100 may move (and about which the drive screw 66 may rotate). Another characterization of the coupled or coupling position is that the ram couplers 158 are positioned so as to be at least partially aligned with the head 38 of the syringe plunger coupler 34 for the corresponding syringe plunger 32. “Alignment” in this regard means in a dimension that is collinear with or parallel to the axis 68.

The various ram couplers 158 are positioned in FIG. 4B to allow the syringe plunger coupler 34 to be uncoupled from the outer ram 140. This may be referred to as the uncoupled or uncoupling position for the ram couplers 158. One characterization of the uncoupled or uncoupling position is that the ram couplers 158 are moved at least generally away from the axis 68 along which the ram assembly 110 may move (and about which the drive screw 66 may rotate). Another characterization of the uncoupled or uncoupling position is that the ram couplers 158 are positioned so as to be out of alignment with the head 38 of the syringe plunger coupler 34 for the corresponding syringe plunger 32. “Alignment” in this regard again means in a dimension that is collinear with or parallel to the axis 68.

The motive force for rotating the inner ram 120 relative to the outer ram 140 is the rotation of the drive screw 66 and the engagement of its helical threads 70 with the threads of the inner ram collar 112 (and on which the inner ram 120 is mounted in the illustrated embodiment). The ram couplers 158 may be disposed in the FIG. 4A position when the ram assembly 110 is in a fully retracted position. When the drive screw 66 is thereafter rotated in a rotational direction that is associated with a discharge stroke for the ram assembly 110 (e.g., in a direction that will advance the corresponding syringe plunger 32 in a direction to provide a discharge from the corresponding syringe 28 (e.g., FIG. 1)), it should be noted that the initial rotation of the drive screw 66 may not actually axially advance the ram assembly 110 along the drive screw 66. This is because the inner ram 120 is allowed to rotate with the drive screw 66 until the pin 148 fixed to the inner ram 120 engages an end of the rotation limit slot 146 on the outer ram 140. It may be desirable to account for this in software that may be used to control one or more aspects of the operation of the power injector 10 (FIG. 1). Rotation of the inner ram 120 in the noted manner responsively moves the ram couplers 158 from the FIG. 4A position to the FIG. 4B position. Therefore and for the case of the discharge stroke of the ram assembly 110, the inner ram end 120 engages (e.g., butts up against) the end of the head 38 of the corresponding syringe plunger coupler 34 to advance the corresponding syringe plunger 32 toward the discharge end 30a of its corresponding syringe 28 (FIG. 1) (e.g., to provide a discharge from the corresponding syringe 28). The syringe 28 could then be removed from the powerhead 12 (FIG. 1) at the end of a discharge stroke without having to first retract the ram assembly 110.

A new, empty syringe 28 may be installed on the powerhead 12 (FIG. 1) with the ram assembly 110 in its fully extended position. When the drive screw 66 is thereafter rotated in a rotational direction that is associated with a retraction stroke for the ram assembly 110 (e.g., in a direction that will advance the corresponding syringe plunger 32 in a direction that is at least generally away from its corresponding discharge end 30a of the syringe 28), it should be noted that the initial rotation of the drive screw 66 may not actually axially advance the ram assembly 110 along the drive screw 66. This again is because the inner ram 120 is allowed to rotate with the drive screw 66 until the pin 148 fixed to the inner ram 120 engages the opposite end of the rotation limit slot 146 on the outer ram 140. It may be desirable to account for this in software that may be used to control one or more aspects of the operation of the power injector 10 (FIG. 1), for instance if the retraction stroke is being used to draw a desired volume of fluid into the syringe 28. Rotation of the inner ram 120 in the noted manner responsively moves the ram couplers 158 from the FIG. 4B position back to the FIG. 4A position. Axial retraction of the ram assembly 110 along the drive screw 66 during continued rotation of the drive screw 66 will cause the ram couplers 158 to “pull” on the head 38 of the syringe plunger coupler 36 for the corresponding syringe plunger 34.

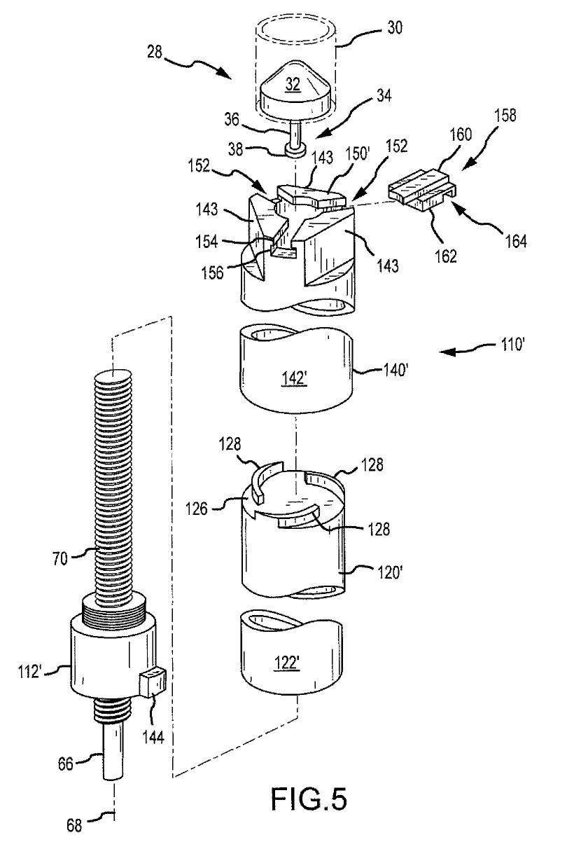

A variation of the ram assembly 110 of FIG. 3A is presented in FIG. 5. Corresponding components between these two embodiments are identified by the same reference numeral, and the discussion presented herein will pertain to each embodiment unless otherwise noted. Those corresponding components that differ in at least some respect are identified by a “single prime” designation in the embodiment of FIG. 5, and will be addressed in relation to any such differences. Notwithstanding the existence of at least certain distances between the two noted embodiments, the various ram couplers 158 utilized by the ram assembly 110′ of FIG. 5 are still at least generally actuated in the same manner that was discussed above in relation to the embodiment of FIG. 3A. The inner ram 120′ includes one or more cams 128 that each positioned within a cam slot 164 on the base 162 of a corresponding ram coupler 158, where each ram coupler 158 is slidably disposed with a coupler slot 152 formed on the outer ram end 150′ of the outer ram 140′. Relative rotation between the inner ram 120′ and the outer ram 140′ causes the ram couplers 158 to move along their respective coupler slots 152 by the interface between their corresponding cam 128 and cam slot 164.

The ram assembly 110′ of FIG. 5 may be used by the power injector 10 of FIG. 1 in place of the ram 20, as well as by the power injector 40 of FIG. 2A in place of the ram 74. However, it should be appreciated that the ram assembly 110′ could of course be utilized by any appropriate power injector, and where this power injector may be utilized for any appropriate application. Hereafter, the ram assembly 110′ will be described in relation to being incorporated by the power injector 10 of FIG. 1.

One difference between the embodiments of FIGS. 3A and 5 is the component of the ram assembly that is rotated to actuate one or more ram couplers 158. The outer ram 140′ in the case of the ram assembly 110′ of FIG. 5 is rotated to move the various ram couplers 158 between a coupled/coupling position and an uncoupled/uncoupling position. In contrast, the inner ram 120 in the case of the ram assembly 110 of FIG. 3A is rotated to move the various ram couplers 158 between a coupled/coupling position and an uncoupled/uncoupling position. Various changes are incorporated into the ram assembly 110′ of FIG. 5 to have the outer ram 140′ rotate to actuate the various ram couplers 158. The ram assembly 110′ of FIG. 5 is mounted on a collar 112′, that in turn is mounted on a drive screw 66 having helical threads 70. Instead of the anti-rotation key 144 being mounted on the outer ram 140 as in the case of the embodiment of FIG. 3A, the ram assembly 110′ has the anti-rotation key 144 mounted or otherwise interconnected with the collar 112′. Since the inner ram 120′ is mounted on the collar 112′ in the above-noted manner, rotation of the drive screw 66 will axially advance the inner ram 120′. However, the inner ram 120′ will not rotate relative to the drive screw 66. Stated another way, the motion of the inner ram 120′ will be primarily limited to axial motion along the drive screw 66 in the case of the ram assembly 110′ of FIG. 5. Any way of maintaining the inner ram 120′ in a rotationally stationary position may be utilized by the ram assembly 110′.

Other changes are incorporated into the ram assembly 110′ of FIG. 5 to have the outer ram 140′ rotate to actuate the various ram couplers 158. The sidewall 142′ of the outer ram 140′ includes one or more coupling elements 143 in the form of a flat surface, and which are used to rotate the outer ram 140′ in a manner that will be discussed in more detail below. These coupling elements 143 change the profile of the outer ram end 150′ in the illustrated embodiment (and thereby the “single prime” designation is used). The ram assembly 110′ may no longer need to incorporate a mechanism to limit the amount that the inner ram 120′ and outer ram 140′ can rotate relative to each other based upon the way in which the outer ram 140′ may be rotated. In this regard, the following structures that were discussed above in relation to the ram assembly 110 of FIG. 3A need not be included in the ram assembly 110′ of FIG. 5: the aperture 124 on the sidewall 122′ of the inner ram 120′; the rotation limit slot 146 on the sidewall 142′ of the outer ram 140′; and the pin 148 that was disposed in the aperture 124 and that traveled within the rotation limit slot 146.

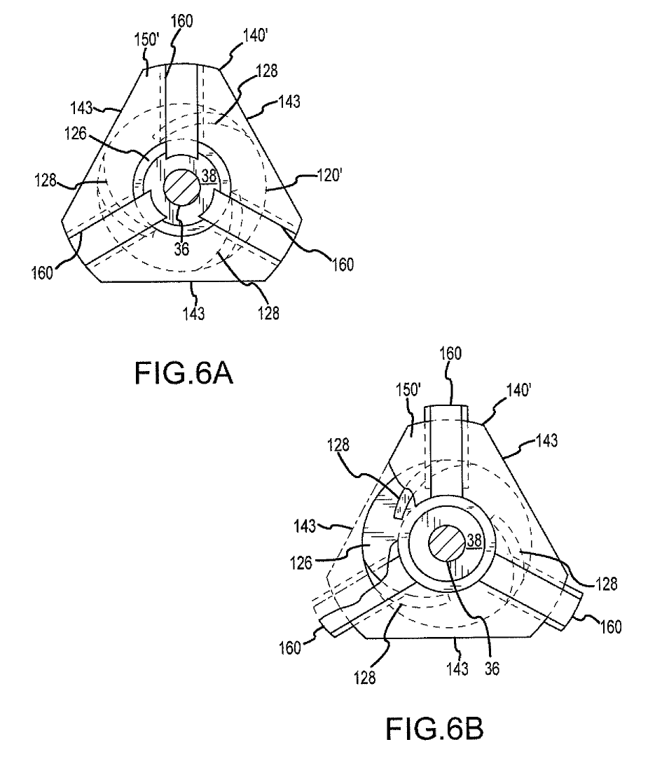

Another difference between the embodiments of FIGS. 3A and 5 is the position of the ram couplers 158 during a certain advancement of the ram assembly 110′ along the drive screw 66. FIG. 6A illustrates the coupled or coupling position for the ram couplers 158 utilized by the ram assembly 110′. The ram couplers 158 may be in the coupled/coupling position of FIG. 6A when the ram assembly 110′ is in its retracted position and when a syringe 28 is installed on the power head 12. Advancing the ram assembly 110′ along the rotating drive screw 66: 1) brings the coupler sections 160 of the ram couplers 158 into contact with the head 38 of the syringe plunger coupler 34 for the corresponding syringe 28; 2) then deflects the coupler sections 160 so that they are now to positioned on the side of the head 38 from which the shaft 36 extends for the corresponding syringe 28 (e.g., the coupler sections 160 “snap” over the head 38 of the associated syringe plunger coupler 34); and 3) then engages the inner ram end 126 with the head 38 of the syringe plunger 32 (e.g., by butting up to the same) to advance the syringe plunger 32 toward the discharge end 30a of the syringe 28 (FIG. 1). At the end of the discharge stroke for the ram assembly 110′, the ram couplers 158 may remain in the FIG. 6A position to retract the syringe plunger 32 as the ram assembly 110′ is retracted along the rotating drive screw 66.

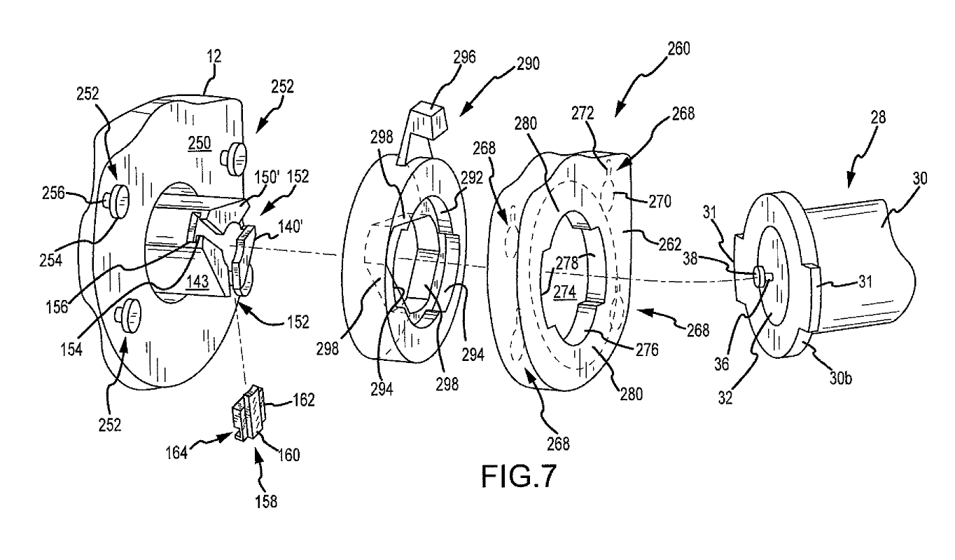

Another difference between the embodiments of FIGS. 3A and 5 is how part of the ram assembly 110′ is rotated to move one or more ram couplers 158 between a coupled/coupling position and an uncoupled/uncoupling position. As discussed above, the rotation of the drive screw 66 itself provides the motive force to rotate the inner ram 120 relative to the outer ram 140 to actuate the various ram couplers 158 in the case of the embodiment of FIG. 3A. FIG. 6A again illustrates the coupled/coupling position for the ram couplers 158 of the ram assembly 110′ of FIG. 5. FIG. 6B illustrates the uncoupled/uncoupling position for the ram couplers 158 of the ram assembly 110′ of FIG. 5. One way for rotating the outer ram 140′ to move the ram couplers 158 between the positions of FIGS. 6A and 6B is illustrated in FIG. 7. Other configurations may be appropriate for rotating the outer ram 140′.

The powerhead 12 from the power injector 10 of FIG. 1 may include various features to incorporate the ram assembly 110′, and which are illustrated in FIG. 7. The powerhead 12 includes an end or a face 250 that projects toward the syringe 28 when installed on the powerhead 12. A plurality of guide pins 252 are disposed in any appropriate arrangement on, are fixed relative to, and extend from the powerhead end 250. Any appropriate number of guide pins 252 may be utilized, and each of the guide pins 252 may be of any appropriate size, shape, and/or configuration. Each guide pin 252 includes a head 254 and a shaft 256 that extends from its corresponding head 254 to the powerhead end 250. Each head 254 is larger than its corresponding shaft 256 in at least one dimension. The ram assembly 110′ of FIG. 5 extends through the powerhead end 250 and is movable relative to the powerhead end 250 along axis 68 (e.g., FIG. 5).

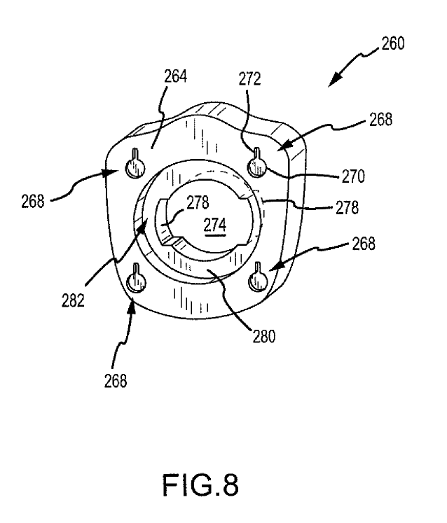

The housing 24 of the power injector 10 of FIG. 1 may be in the form of a faceplate 260, which may be installed on the powerhead end 250 utilizing the guide pins 252, and is illustrated in FIG. 7 as well as FIG. 8. The faceplate 260 includes opposing end surfaces 262 and 264. The end surface 264 projects toward the powerhead end 250 when the faceplate 260 is installed on the powerhead 12. The end surface 262 is oppositely disposed, and projects in the direction of the discharge end 30a of the syringe 28 when the syringe 28 is installed on the powerhead 12 using the faceplate 260.

The end surface 264 of the faceplate 260 includes a plurality of guide pin slots 268, and these slots 268 do not extend entirely through the faceplate 268. There is one guide pin slot 268 on the faceplate 260 for each guide pin 252 on the powerhead end 250. Each guide pin slot 268 includes a head section slot 270 and a shaft section slot 272. The head section slot 270 is sized to receive the head 254 of the corresponding guide pin 252 on the powerhead end 250, and the head section slot 270 extends upwardly behind the shaft section slot 272 in the views shown in FIGS. 7 and 8. The shaft section slot 272 is sized to receive the shaft 256 of the corresponding guide pin 252 on the powerhead end 250, but is smaller than the head 254 of the corresponding guide pin 252 on the powerhead end 250. As such, the head 254 of the corresponding guide pin 252 on the powerhead end 250 may be positioned within the portion of the head section slot 270 that extends below the shaft section slot 272. Thereafter, the faceplate 260 may be moved relative to the powerhead 12 within a plane that is orthogonal to the axis 68 (along which the ram assembly 110′ may move) to dispose each head 254 within a portion of its corresponding head section slot 270 that is behind the corresponding shaft section slot 272. Movement of the faceplate 260 relative to the powerhead 12 will now be restrained along the axis 68 (along which the ram assembly 110′ may move). Notwithstanding the foregoing, it should be appreciated that the faceplate 260 may be mounted on the powerhead end 250 in any appropriate manner, including simply by using one or more threaded fasteners.

An aperture 274 extends entirely through the faceplate 260 progressing from one end surface 262 to the opposite end surface 264. This aperture 274 is sized to allow the outer ram 140′ to proceed through the faceplate 260 when the outer ram 140′ is being directed into the barrel 30 of the syringe 28 on a discharge stroke. A perimeter of this aperture 274 is defined by an inner wall 276. One or more locking or coupling element slots 278 are formed in this inner wall 276. One or more coupling elements or locking sections 280 are also disposed about the aperture 274. The locking slots 278 and the locking sections 280 are used to detachably interconnect the syringe 28 with the faceplate 260 in a manner that will be discussed in more detail below.

A cam plate 290 is movably disposed within a cam plate recess 282 formed on the end surface 264 of the faceplate 260, which again projects toward the powerhead end 250 when the faceplate 260 is installed on the powerhead 12. An aperture 292 extends entirely through the cam plate 290 to accommodate a coupling of the ram assembly 110′ with the syringe plunger coupler 34. The end portion of the aperture 292 that projects toward the syringe 28 is larger than the end portion of the aperture 292 that projects toward the powerhead end 250. This allows the outer ram 140′ to proceed through the cam plate 290 when the outer ram 140′ is being directed into the barrel 30 of the syringe 28 on a discharge stroke.

One or more coupling elements in the form of slots 294 are included on the cam plate 290 for interfacing with a corresponding coupling element or flange segment 31 on the flange 30b of the syringe 28. A plurality of coupling elements 298 may be formed into or otherwise integrated with the cam plate 290 and interface with a corresponding coupling element 143 on the perimeter of the outer ram 140′. In the illustrated embodiment, the plurality of coupling elements 298 are in the form of one or more surfaces on the perimeter of the end portion of the aperture 292 of the cam plate 290 that projects toward the powerhead end 250 and into which the outer ram 140′may extend. The entire perimeter of this end portion of the aperture 292 of the cam plate 290 could at least generally match and at least generally engage the entire perimeter of the outer ram 140′. Any appropriate way of “keying” the outer ram 140′ to the cam plate 290 may be utilized such that the outer ram 140′ is able to rotate along with the cam plate 290.

The cam plate 290 may be moved relative to the powerhead 12 within the cam plate recess 282 of the faceplate 260 in any appropriate manner. A handle 296 may be fixed relative to the cam plate 290 to facilitate movement of the cam plate 290 within the cam plate recess 282 of the faceplate 260, although such may not be required in all instances (e.g., the syringe 28 could be utilized to move the cam plate 290 within the cam plate recess 282 of the faceplate 260). When the cam plate coupling elements 298 interface with the outer ram coupling elements 143, the cam plate 290 may be used to control the rotational position of the outer ram 140′. It should be appreciated that the cam plate coupling elements 298 and the outer ram coupling elements 143 each may be of any appropriate size, shape, configuration, and/or type. Any appropriate number of cam plate coupling elements 298 and outer ram coupling elements 143 may be utilized. Any way of coupling the cam plate 290 and/or syringe 28 with the outer ram 140′ may be utilized to allow the cam plate 290 and/or the syringe 28 to be used to control the rotational position of the outer ram 140′.

The installation of the syringe 28 on the powerhead 12 of FIG. 7 will now be described. The faceplate 260 will have been positioned on and supported by the powerhead 12 in the above-noted manner. The syringe 28 may be advanced relative to the faceplate 260 with its coupling elements 31 being aligned with and passing through the locking slots 278 on the faceplate 260. The syringe 28 may continue to be advanced until each coupling element 31 is disposed within its corresponding coupling element slot 294 on the cam plate 290. At this time, the head 38 of the syringe plunger 32 may have passed through the space collectively defined by the various ram couplers 158, and thereby may be disposed within the outer ram 140′. The syringe 28 may now be rotated relative to the powerhead 12. This relative rotational movement may be achieved in any appropriate manner, including by rotating the cam plate 290 via the handle 296 (which will rotate the syringe 28 at least generally about its long axis, and which may coincide with the axis 68 along which the ram assembly 110′ may move and about which the drive screw 66 may rotate), by rotating/turning the syringe 28 at least generally about its long axis (which will rotate the cam plate 290 within the cam plate recess 282 on the faceplate 260), or both.

Relative movement between the cam plate 290/syringe 28 and the faceplate 260 locks the syringe 28 to the faceplate 260. Specifically, the coupling elements 31 on the syringe flange 30b will be moved into at least partial alignment with the locking sections 280 of the faceplate 260. The outer ram 140′ will also move along with the cam plate 290 and syringe 28. This movement of the outer ram 140′ is a rotational movement about the axis 68 along with the ram assembly 110′ may move and about which the drive screw 66 may rotate, and is relative to the inner ram 120′ which remains in a rotationally stationary position at this time. Rotation of the outer ram 140′ moves the ram couplers 158 relative to the outer ram end 150. Again, the ram couplers 158 are carried by the outer ram 140′, and each has a cam slot 164 that interfaces with a cam 128 on the inner ram 120′. Since there is relative rotational movement between the outer ram 140′ and the inner ram 120, the cams 128 move within their corresponding cam slot 164 to move the ram couplers 158 relative to the ram outer end 150′.

It should be appreciated that the syringe plunger coupler 34 may be uncoupled from the ram couplers 158 of the outer ram 140′ by reversing the above-noted protocol. It should also be appreciated that the cam plate 290 may be locked in any appropriate manner in one of or each of its two end positions as desired/required—one end position of the cam plate 290 being where the various ram couplers 158 are disposed in the FIG. 6A position, and another end position of the cam plate 290 being where the various ram couplers 158 are disposed in the FIG. 6B position. The cam plate 290 could also be biased to at least one of its end positions in any appropriate manner.

The ram assembly 110 of FIG. 3A uses the rotation of the drive screw 66 to rotate the inner ram 120 relative to the rotationally stationary outer ram 140, which in turn actuates the various ram couplers 158 (e.g., moves the ram couplers 150 relative to the outer ram end 150). The ram assembly 110′ of FIG. 5 uses the rotation of the cam plate 290 and/or syringe 28 to rotate the outer ram 140′ relative to the rotationally stationary inner ram 120′. Each of these ram assemblies 110, 110′ could use any appropriate mechanism for providing relative rotational movement between their respective inner and outer rams. Each of these ram assemblies 110, 110′ could also use two independently operable drive sources of sorts for providing relative rotational movement between their respective inner and outer rams. For instance, each of the ram assemblies 110, 110′ could rotate their respective inner ram 120 and outer ram 140′ in the manner discussed herein, along with another appropriate mechanism to rotate their respective inner and outer rams relative to each other and at any appropriate time (e.g., switch-activated). Although only one of the inner and outer rams is rotated to actuate the ram couplers 158 in each of the above-noted embodiments, it should be appreciated that each of the inner and outer rams could be rotated to provide the desired relative rotational movement.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

1. A power injector comprising a rotatable drive screw and a ram assembly mounted on said drive screw, said ram assembly comprising:
a first ram section comprising an inner ram;
a second ram section comprising an outer ram that is movable relative to said first ram section, wherein said first and second ram sections are collectively movable along said drive screw in each of first and second directions during rotation of said drive screw in first and second rotational directions, respectively; and
a ram coupler movable relative to at least one of said first and second ram sections responsively to a relative movement between said first and second ram sections, wherein a movement of said ram coupler relative to said at least one of said first and second ram sections establishes at least one of a coupling and an uncoupling position for said ram coupler in relation to a syringe plunger coupler of a syringe, and wherein said ram assembly is movable along a path to discharge fluid from the syringe.

2. The power injector of claim 1, wherein said
outer ram of said second ram section movable relative to said inner ram and comprises an outer ram end that defines an end of said ram assembly, and wherein said
ram coupler is slidably interconnected with said outer ram end and movable relative to said outer ram responsively to a relative movement between said inner and outer rams.

3. The power injector of claim 1, wherein said second ram section is disposed about at least part of said first ram section.

4. The power injector of claim 1, wherein said first and second ram sections are concentrically disposed.

5. The power injector of claim 1, wherein said first ram section, said second ram section, and said ram coupler are collectively movable along an axial path.

6. The power injector of claim 1, wherein said first and second ram sections are rotatable relative to each other.

7. The power injector of claim 1, wherein a relative movement between said first and second ram sections actuates said ram coupler.

8. The power injector of claim 1, wherein a relative movement between said first and second ram sections causes said ram coupler to move relative to each of said first and second ram sections.

9. The power injector of claim 1, wherein a relative movement between said first and second ram sections is of a first type and wherein a movement of said ram coupler relative to said second ram section is of a second type that is different from said first type.

10. The power injector of claim 1, wherein said first ram section comprises an inner ram, wherein said second ram section comprises an outer ram, wherein said inner ram is at least partially disposed within said outer ram, wherein said outer ram comprises an outer ram end that defines an end of said ram assembly, wherein said ram coupler is slidably interconnected with said outer ram end, wherein a rotational lock is engaged with said outer ram, and wherein said inner ram is rotated to move said ram coupler relative to said outer ram end and while said outer ram is maintained at least generally in a rotationally stationary position by said rotational lock.

11. The power injector of claim 10, further comprising a rotational range limiter interconnected with said inner ram.

12. The power injector claim 10, wherein said outer ram comprises a slot, wherein a stop extends through said slot to said inner ram and is maintained in a fixed position relative to said inner ram, wherein said stop moves along said slot during a rotation of said inner ram, and wherein a length of said slot defines a range through which said inner ram may rotate.

13. The power injector of claim 1, wherein said first ram section comprises an inner ram, wherein said second ram section comprises an outer ram, wherein said inner ram is at least partially disposed within said outer ram, wherein said outer ram comprises an outer ram end that defines an end of said ram assembly, wherein said ram coupler is slidably interconnected with said outer ram end, wherein a rotational lock is interconnected with said inner ram, and wherein said outer ram is rotated to move said ram coupler relative to said outer ram end and while said inner ram is at least generally maintained in a rotationally stationary position by said rotational lock.

14. The power injector of claim 1, wherein said ram coupler is slidably interconnected with one of said first and second ram sections, and wherein said ram coupler is movable along an axial path relative to said one of said first and second ram sections.

15. The power injector of claim 1, wherein said first ram section comprises an inner ram, wherein said second ram section comprises an outer ram, wherein said inner ram is at least partially disposed within said outer ram, wherein said outer ram comprises an outer ram end that defines an end of said ram assembly, wherein said outer ram end comprises a slot, and wherein said ram coupler is slidably disposed within said slot.

16. The power injector of claim 1, wherein one of said first and second ram sections comprises a first camming member, wherein said ram coupler comprises a second camming member engageable with said first camming member, and wherein relative movement between said first and second camming members actuates said ram coupler.

17. The power injector of claim 1, wherein said first ram section comprises an inner ram, wherein said second ram section comprises an outer ram, wherein said inner ram is at least partially disposed within said outer ram, wherein said outer ram comprises an outer ram end that defines an end of said ram assembly, wherein said ram coupler is slidably interconnected with said outer ram end, wherein said inner ram comprises a first camming member, wherein said ram coupler comprises a second camming member engageable with said first camming member, and wherein relative movement between said first and second camming members actuates said ram coupler.

18. The power injector of claim 1; wherein said ram coupler is disposed so that a head of the syringe plunger coupler is in a captured state during a retraction of said ram assembly.

19. The power injector of claim 1, wherein said ram coupler is disposed so that a head of the syringe plunger coupler is in a captured state during an extension of said ram assembly for a syringe discharge stroke.

20. The power injector of claim 1, wherein said ram coupler is disposed so that an end of said first ram section engages an end of a head of the syringe plunger coupler during an extension of said ram assembly for a syringe discharge stroke.

21. The power injector of claim 1, further comprising a plurality of said ram couplers.

22. The power injector of claim 21, wherein said plurality of ram couplers are collectively movable in response to a relative movement between said first and second ram sections.

23. The power injector of claim 21, wherein each of said plurality of ram couplers are movable at least generally away from a common locale, and are also movable at least generally toward said common locale.

24. The power injector of claim 21, wherein each of said plurality of ram couplers is movable along a different axial path relative to at least one of the first and second ram sections.

25. The power injector of claim 1, further comprising:
a powerhead; and
a syringe installed on said powerhead, wherein said powerhead comprises said rotatable drive screw, wherein said first ram section comprises an inner ram, wherein said second ram section comprises an outer ram, wherein said outer ram comprises an outer ram end that defines an end of said ram assembly, and wherein said ram coupler is slidably interconnected with said outer ram end.

26. The power injector of claim 25, wherein said inner ram comprises a first camming member, wherein said ram coupler comprises a second camming member engageable with said first camming member, and wherein relative movement between said first and second camming members actuates said ram coupler.

27. A method for changing a coupled state between a syringe plunger and a ram assembly of a power injector, wherein a syringe comprises a syringe plunger and a syringe plunger coupler interconnected with said syringe plunger, wherein said ram assembly comprises a first ram section, a second ram section, and a ram coupler, and wherein said method comprises the steps of:
installing said syringe on said power injector;
rotating said first ram section relative to said second ram section after said installing step;
moving said ram coupler relative to at least one of said first ram section and said second ram section responsively to said rotating step; and
changing a coupled state between said syringe plunger coupler and said ram coupler as a result of said moving said ram coupler step.

28. (canceled)

29. The method of claim 27, wherein said first ram section comprises an inner ram, wherein said second ram section comprises an outer ram, wherein said inner ram is at least partially disposed within said outer ram, wherein said outer ram comprises an outer ram end that defines an end of said ram assembly, and wherein said ram coupler is slidably interconnected with said outer ram end.

30. The method of claim 29, wherein said rotating step comprises rotating said inner ram.

31. The method of claim 30, further comprising the step of limiting a rotational range for said rotating said inner ram step.

32. The method of claim 30, further comprising the steps of rotating a drive screw and axially advancing said ram assembly along said drive screw during said rotating a drive screw step, wherein said rotating said inner ram step is executed in response to said rotating a drive screw step.

33. The method of claim 30, further comprising the steps of rotating a drive screw and axially advancing said ram assembly along said drive screw during said rotating a drive screw step, wherein a primary force for said rotating said inner ram step comprises a threaded engagement between said drive screw and said ram assembly, along with a force generated during said rotating a drive screw step based upon said threaded engagement.

34. The method of claim 29, wherein said rotating step comprises rotating said outer ram.

35. The method of claim 34, further comprising the step of rotating said syringe, wherein said rotating said outer ram step is responsive to said rotating a syringe step.

36. The method of claim 34, further comprising the steps of providing a coupling between said syringe and said outer ram, and thereafter rotating said syringe, wherein said rotating said outer ram step is responsive to said rotating a syringe step.

37. The method of claim 29, wherein said moving said ram coupler step comprises camming said ram coupler off of said inner ram during at least part of said rotating step.

38. The method of claim 29, wherein said ram coupler is slidably interconnected with said outer ram, wherein said moving said ram coupler step comprises moving said ram coupler along an axial path relative to said outer ram.

39. The method of claim 27, wherein said ram assembly further comprises a plurality of said ram couplers.

40. The method of claim 39, further comprising the step collectively moving said plurality of said ram couplers to a coupling position in relation to said syringe plunger coupler, and collectively moving said plurality of said ram couplers to an uncoupling position in relation to said syringe plunger coupler.

41. The method of claim 40, wherein said collectively moving said plurality of said ram couplers to a coupling position step comprises moving said plurality of ram couplers at least generally toward a common locale, and wherein said collectively moving said plurality of ram couplers to an uncoupling position comprises moving said plurality of said ram couplers at least generally away from said common locale.

42. The method of claim 27, wherein said changing a coupled state step comprises capturing said syringe plunger coupler of said syringe, wherein said method further comprises the steps of:
retracting said ram assembly after said capturing step; and
retracting said syringe plunger of said syringe in response to said retracting said ram assembly step.

43. The method of claim 42, further comprising the step of:
extending said ram assembly after said capturing step, wherein said syringe plunger coupler remains captured during said extending step, and wherein a fluid is discharged from said syringe by said extending step.

44. The method of claim 42, further comprising the step of:
extending said ram assembly after said capturing step, wherein a fluid is discharged from said syringe by said extending step, and wherein said changing a coupled state step comprises changing said ram coupler from a coupled state to an uncoupled state relative to said syringe plunger coupler for said extending step.
